(19) United States
(12) Patent Application Publication
(10) Pub. No.: US 2020/0278343 A1
(43) Pub. Date: Sep. 3, 2020

(54) DIRECT VISUALIZATION OF INTEGRATED STRESS RESPONSE ACTIVITY

(71) Applicants: Board of Supervisors of Louisiana State University and Agricultural and Mechanical College, Baton Rouge, LA (US); University of North Carolina at Chapel Hill, Chapel Hill, NC (US)

(72) Inventors: Jeremy P. KAMIL, Shreveport, LA (US); Nathaniel MOORMAN, Chapel Hill, NC (US); Hongbo ZHANG, Shreveport, LA (US); Christopher NGUYEN, Shreveport, LA (US)

(21) Appl. No.: 16/805,686

(22) Filed: Feb. 28, 2020

Related U.S. Application Data
(60) Provisional application No. 62/811,837, filed on Feb. 28, 2019.

(51) Int. Cl. G01N 33/50; G01N 33/68

(52) U.S. Cl. CPC G01N 33/5091; G01N 33/6893

(57) ABSTRACT

A method of detecting integrated stress response activity in living cells comprising inducing UL148-marker expression, scanning the cells to determine if the UL148 is punctate or diffuse, and ranking the level of ISR activity based on how punctate the UL148 is in the cell. The marker may be a fluorescent marker. The fluorescent marker may be Green fluorescent protein. The cell may be an epithelial cell. The cells may be ARPE-19 epithelial cells carrying a doxycycline inducible gene expression cassette encoding UL148-GFP and treating the cells for 18 hours with 100 ng/mL doxycycline hyclate to induce UL148-GFP expression. The cells may have an inducible gene expression cassette encoding UL148-marker expression. The UL148-marker expression may be induced by the cell being treated with an antibiotic. The method may include adding marker to the cells. The cells may be mutated to express UL148-marker upon inducement. A kit to conduct the method.

### CROSS REFERENCE TO RELATED APPLICATIONS/PRIORITY

The present invention claims priority to United States Provisional Patent Application No. 62/811,837 filed Feb. 28, 2019, which, including its Appendix, is incorporated by reference into the present disclosure as if fully restated herein. Any conflict between the incorporated material and the specific teachings of this disclosure shall be resolved in favor of the latter. Likewise, any conflict between an art-understood definition of a word or phrase and a definition of the word or phrase as specifically taught in this disclosure shall be resolved in favor of the latter.

### STATEMENT REGARDING FEDERALLY SPONSORED RESEARCH OR DEVELOPMENT

This invention was made with government support under Grant No. 1R01A1116851 awarded by the National Institutes of Health. The government has certain rights in the invention.

### BACKGROUND

Integrated Stress Response is of enormous relevance to cancer, diabetes, cardiovascular disease and stroke, infectious diseases and human genetic diseases. Current ISR screens though are complex, and expensive to conduct. For instance, the ISR inhibitor screen used to identify IRSIB, C₂₂H₂₄Cl₂N₂O₄, required engineering cells to express a luciferase protein only under conditions of ER stress. Luciferase screens require more steps and expensive substrates to generate data for interpretation. There is a pressing, but seemingly irresolvable need for a simple, inexpensive ISR screen.

### SUMMARY

Wherefore, it is an object of some embodiments of the present invention to overcome the above-mentioned shortcomings and drawbacks associated with the current technology. The invention relates to devices, cells, kits, and methods of detecting integrated stress response activity in living cells comprising inducing UL148-marker expression, scanning the cells to determine if the UL148 is punctate or diffuse, and ranking the level of ISR activity based on how punctate the UL148 is in the cell. The marker may be a fluorescent marker. The fluorescent marker may be Green fluorescent protein. The cell may be an epithelial cell. The cells may be ARPE-19 epithelial cells carrying a doxycycline inducible gene expression cassette encoding UL148-GFP and treating the cells for 18 hours with 100 ng/mL doxycycline hyclate to induce UL148-GFP expression. The cells may have an inducible gene expression cassette encoding UL148-marker expression. The UL148-marker expression may be induced by the cell being treated with an antibiotic. The method may include adding marker to the cells. The cells may be mutated to express UL148-marker upon inducement.

Various objects, features, aspects, and advantages of the present invention will become more apparent from the following detailed description of preferred embodiments of the invention, along with the accompanying drawings in which like numerals represent like components. The present invention may address one or more of the problems and deficiencies of the current technology discussed above. However, it is contemplated that the invention may prove useful in addressing other problems and deficiencies in a number of technical areas. Therefore the claimed invention should not necessarily be construed as limited to addressing any of the particular problems or deficiencies discussed herein.

### BRIEF DESCRIPTION OF THE DRAWINGS

The accompanying drawings, which are incorporated in and constitute a part of the specification, illustrate various embodiments of the invention and together with the general description of the invention given above and the detailed description of the drawings given below, serve to explain the principles of the invention. It is to be appreciated that the accompanying drawings are not necessarily to scale since the emphasis is instead placed on illustrating the principles of the invention. The invention will now be described, by way of example, with reference to the accompanying drawings in which:

FIG. 1 is a set of photomicrographs showing UL148 causes formation of unusual ER structures during HCMV infection;

FIG. 2 is a pair of photomicrographs showing UL148 fused to GFP is a visual probe for ISR inhibition in living cells; and

FIG. 3 is an example of current ISR screen technology.

### DETAILED DESCRIPTION

The present invention will be understood by reference to the following detailed description, which should be read in conjunction with the appended drawings. It is to be appreciated that the following detailed description of various embodiments is by way of example only and is not meant to limit, in any way, the scope of the present invention. In the summary above, in the following detailed description, in the claims below, and in the accompanying drawings, reference is made to particular features (including method steps) of the present invention. It is to be understood that the disclosure of the invention in this specification includes all possible combinations of such particular features, not just those explicitly described. For example, where a particular feature is disclosed in the context of a particular aspect or embodiment of the invention or a particular claim, that feature can also be used, to the extent possible, in combination with and/or in the context of other particular aspects and embodiments of the invention, and in the invention generally. The term “comprises” and grammatical equivalents thereof are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also one or more other components. Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range beginning with that number (which may be a range having an upper limit or no upper limit, depending on the variable being defined). For example “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40% means 40% or less than 40%. When, in this specification, a range is given as “(a first number) to (a second number)” or “(a first number)−(a second number),” this means a range whose lower limit is the first number and whose upper limit is the second number. For example, 25 to 100 mm means a range whose lower limit is 25 mm, and whose upper limit is 100 mm. The embodiments set forth the below represent the necessary information to enable those skilled in the art to practice the invention and illustrate the best mode of practicing the invention. In addition, the invention does not require that all the advantageous features and all the advantages need to be incorporated into every embodiment of the invention.

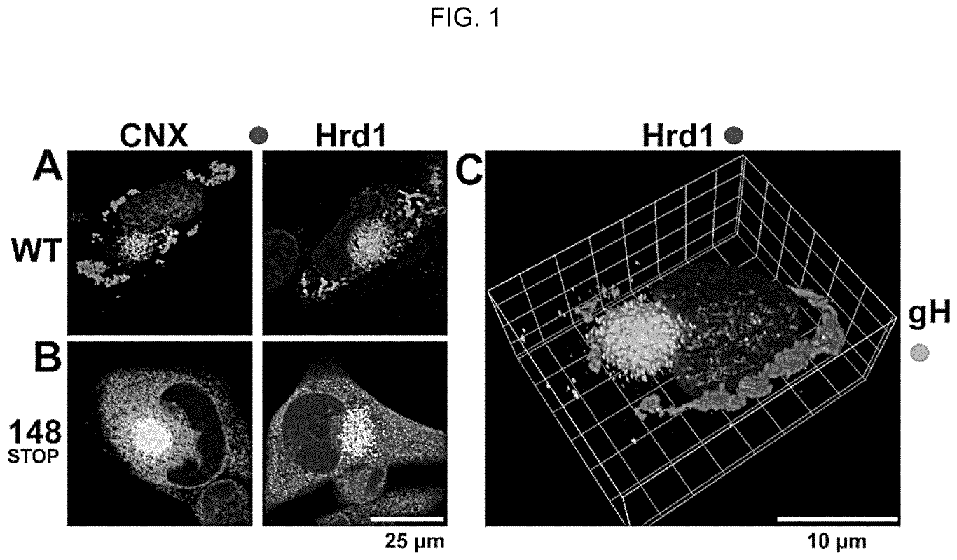

Turning now to FIGS. 1 to 3, a brief description concerning the various components of the present invention will now be briefly discussed.

The inventors have developed a novel and inexpensive technology to visualize whether the Integrated Stress Response is active in living cells. This technology will likely be valuable for commercial efforts to identify new drugs that inhibit the ISR, and hence which may represent leads for development of novel therapeutic agents. In brief, the disclosed technology will enable inexpensive screening platforms to rapidly identify novel small molecule inhibitors of the ISR.

The disclosed technology is based on UL148, a viral endoplasmic reticulum (ER)-resident protein, which influences the expression of a viral glycoprotein complex required for cell entry. UL148 activates the Unfolded Protein Response (UPR), resulting in stress-regulated translation of ISR mRNAs such as ATF4. ISR/UPR activation by UL148 causes the formation of large membranous structures from the ER, at which UL148 itself accumulates. These structures occupy 2-7% of the cytosol, and hence are visually prominent features of cells expressing UL148. Furthermore, when UL148 is fused at its carboxy-terminus to a fluorescent protein, such as green fluorescent protein (GFP—a protein composed of 238 amino acid residues (26.9 kDa) that exhibits bright green fluorescence when exposed to light in the blue to ultraviolet range), and expressed in living cells, the formation of these ER structures can be visualized in real-time. Other fluorescent proteins may include Allophycocyanin, Brainbow, Cameleon, FMN-binding fluorescent protein, Glutamate-sensitive fluorescent reporter, MCherry, Phycoerythrin, Red fluorescent protein, RoGFP, SmURFP, Synapto-pHluorin, and Yellow fluorescent protein. ISRIB, a highly efficacious small molecule inhibitor of the ISR completely blocks the formation of UL148 structures. Instead, treatment with an ISR-inhibitor (e.g., ISRIB) causes UL148-GFP to be evenly distributed across the ER such that the GFP staining is uniform and reticular. Therefore, the simple task of determining whether GFP signal is present and whether it is uniform or punctate provides a facile read-out that can be automated to enable high throughput screening for novel small molecule inhibitors that inhibit the ISR.

As shown in FIG. 1, UL148 causes formation of unusual ER structures during HCMV infection. Wild-type (WT) and UL148-null mutant (148STOP) human cytomegalovirus (HCMV) were used to infect fibroblasts. Images are from day 4 post infection. Signal from antibodies specific for the ER markers calnexin (CNX) and Hrd1, and of the viral glycoprotein H (gH) are shown in stained, fixed, cells. FIG. 1A shows WT infection, FIG. 1B shows 148STOP infection, and FIG. 1C shows 3D image of Hrd1 and HCMV glycoprotein H (gH) during WT infection.

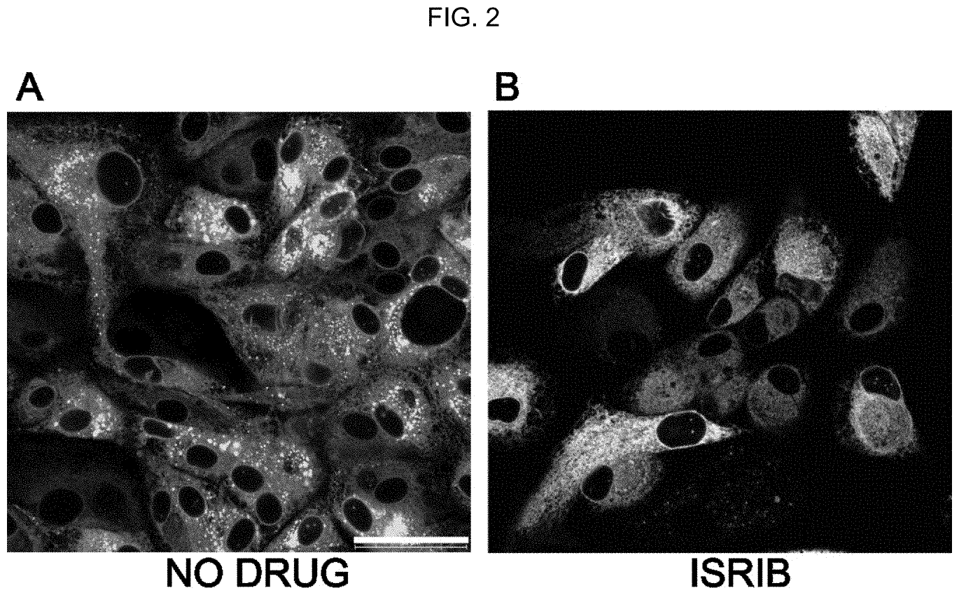

As shown in FIG. 2, UL148 fused to GFP is a visual probe for ISR inhibition in living cells. ARPE-19 epithelial cells carrying a doxycycline inducible gene expression cassette encoding UL148-GFP were treated for 18 hours with 100 ng/mL doxycycline hyclate to induce UL148-GFP expression. In panel A, (FIG. 2A) UL148-GFP was induced in the absence of ISRIB. In panel B, (FIG. 2B) induction of UL148-GFP was carried out in the presence of 200 nM ISRIB, an ISR inhibitor.

The technology described herein can be used by biotech and biopharma firms that wish to screen small molecule libraries to identify ISR inhibitors/lead compounds to eventually develop into FDA-approved therapeutic drugs to inhibit the ISR, for example to treat diseases associated with inappropriate or excessive ISR activation.

Various embodiments of the disclosed invention have strong competitive advantages over the current technology. The use of UL148-fused to GFP (or similar fluorescent protein tags) to directly screen for ISR inhibitors is less complex and more straightforward than current “best-practices” for ISR inhibitors. The inventors' assay would preferably use an automated system to interpret punctate versus diffuse GFP signal in cells induced to express UL148-GFP. In short, the inventors' invention provides a substantial cost advantage over the competition and likely will prove to be more robust and accurate method to identify ISR inhibitors.

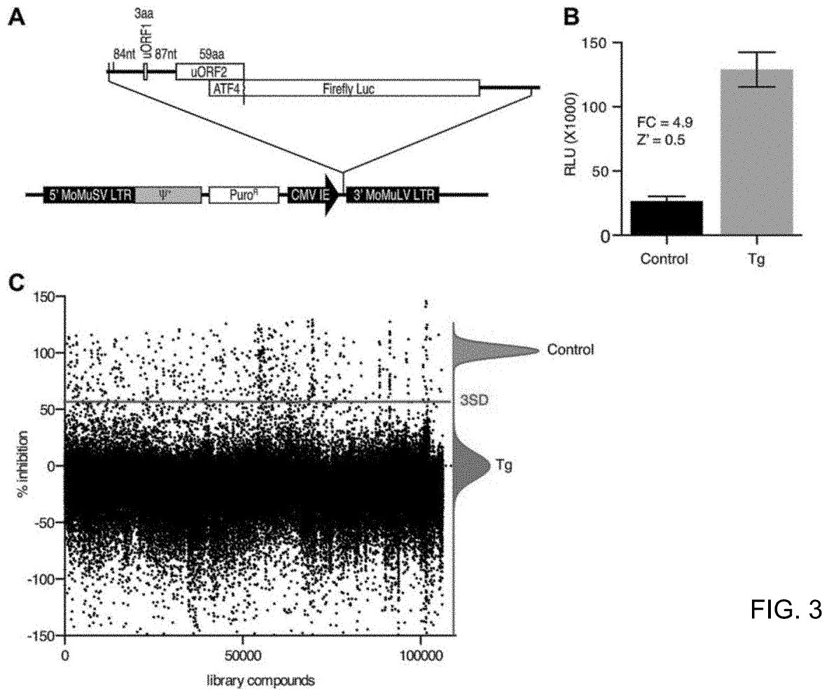

As shown in FIG. 3 current ISR screen technology does not convey the advantages of embodiments of the presently claimed invention. The is from Sidrauski et al. eLife 2013; 2:e00498. DOI: 10.7554/eLife.00498.

Eukaryotic cells are equipped with three sensors that respond to the accumulation of misfolded proteins within the lumen of the endoplasmic reticulum (ER) by activating the unfolded protein response (UPR), which functions to resolve proteotoxic stresses involving the secretory pathway. Here, the inventors identify UL148, a viral ER-resident glycoprotein from human cytomegalovirus (HCMV), as an inducer of the UPR. Metabolic labeling results indicate that global mRNA translation is decreased when UL148 expression is induced in uninfected cells. Further, the inventors find that ectopic expression of UL148 is sufficient to activate at least two UPR sensors: the inositol-requiring enzyme-1 (IRE1), as indicated by splicing of Xbp-1 mRNA, and the protein kinase R (PKR)-like ER kinase (PERK), as indicated by phosphorylation of the α subunit of eukaryotic initiation factor 2 (eIF2α) and accumulation of activating transcription factor 4 (ATF4). During wild-type HCMV infection, increases in Xbp-1 splicing, eIF2a phosphorylation, and accumulation of ATF4 accompany UL148 expression. UL148-null infections, however, show reduced levels of these UPR indicators and decreases in XBP1s abundance and in phosphorylation of PERK and IRE1. Small interfering RNA (siRNA) depletion of PERK dampened the extent of eIF2a phosphorylation and ATF4 induction observed during wild-type infection, implicating PERK as opposed to other eIF2a kinases. A virus with UL148 disrupted showed significant 2- to 4-fold decreases during infection in the levels of transcripts canonically regulated by PERK/ATF4 and by the ATF6 pathway. Taken together, the inventors' results argue that UL148 is sufficient to activate the UPR when expressed ectopically and that UL148 is an important cause of UPR activation in the context of the HCMV-infected cell.

The unfolded protein response (UPR) is an ancient cellular response to ER stress that is of broad importance to viruses. Certain consequences of the UPR, including mRNA degradation and translational shutoff, would presumably be disadvantageous to viruses, while other attributes of the UPR, such as ER expansion and upregulation of protein folding chaperones, might enhance viral replication. Although HCMV is estimated to express well over 150 different viral proteins, the inventors show that the HCMV ER-resident glycoprotein UL148 contributes substantially to the UPR during infection and, moreover, is sufficient to activate the UPR in noninfected cells. Experimental activation of the UPR in mammalian cells is difficult to achieve without the use of toxins. Therefore, UL148 may provide a new tool to investigate fundamental aspects of the UPR. Furthermore, the inventors' findings may have implications for understanding the mechanisms underlying the effects of UL148 on HCMV cell tropism and evasion of cell-mediated immunity.

The endoplasmic reticulum (ER) is a fundamental eukaryotic organelle comprised of a tubulovesicular network of membranes that extends throughout the cytosol. The organelle carries out multifarious processes vital to cellular and organismal health. For instance, the ER plays key roles in the regulation of intracellular calcium levels and provides the site for steroid and lipid synthesis, loading of peptides onto major histocompatibility complex (MHC) complexes, and synthesis and processing of proteins and protein complexes destined for secretion. Therefore, it is no surprise that the ER is exploited by a diverse array of viruses during their replication. For instance, polyomaviruses exploit the ER for entry, whereas flaviviruses and caliciviruses remodel it to creates sites for replication. Large enveloped double-stranded DNA (dsDNA) viruses, such as those in the Herpesviridae, require the ER for the expression and processing of extraordinarily large amounts of viral glycoproteins needed for the assembly of progeny virions.

In order to utilize the ER to support their replication, however, viruses have had to develop mechanisms to contend with the unfolded protein response (UPR), an ancient stress response that serves to maintain ER function and cell viability when misfolded proteins accumulate within the secretory pathway. The UPR is initiated by three different ER-based signaling molecules: inositol-requiring enzyme-1 (IRE1), the protein kinase R (PKR)-like ER kinase (PERK), and the cyclic AMP-dependent transcription factor 6 α (ATF6). Misfolded proteins are thought to displace the ER chaperone BiP (Grp78) from the luminal domains of IRE1, PERK, and ATF6, which causes their activation. During the UPR, mRNA translation is attenuated, transcripts associated with rough ER ribosomes are degraded, and a number of genes are transcriptionally upregulated, resulting in increased expression of ER protein-folding chaperones, ER-associated degradation (ERAD) proteins, and various factors that can expand the size and secretory capacity of the ER. Hence, certain consequences of the UPR, particularly translational attenuation, would be expected to be deleterious to viruses, while others, such as ER expansion, could enhance the capacity of the infected host cell to produce progeny virions.

Cytomegaloviruses have been found to activate the UPR while subverting certain aspects of it. Interestingly, the viral nuclear egress complex component m50 of murine cytomegalovirus (MCMV) degrades IRE1, and the human cytomegalovirus (HCMV) homolog UL50 apparently shares this activity. Cytoplasmic splicing of Xbp-1 mRNA is mediated by IRE1 nuclease activity upon UPR activation. This splicing event is required for translation of the transcription factor XBP1s, which upregulates ERAD factors and ER chaperones, among other target genes. In addition, IRE1 degrades mRNAs undergoing translation at the rough ER. Therefore, IRE1 downregulation may help to maintain viral glycoprotein expression in the face of UPR activation. Despite this function of UL50, Isler et al. found evidence that IRE1 is activated during HCMV infection. In addition to IRE1, PERK is activated during HCMV and MCMV infection, and the PERK/activating transcription factor 4 (ATF4) axis appears to be required for efficient viral replication, as defects in viral upregulation of lipid synthesis are observed in cells lacking PERK.

Notably, the viral proteins or processes that activate PERK and IRE1 in the context of HCMV infection have not been clearly identified. The inventors recently reported that UL148 interacts with SEL1L, a component of the cellular ERAD machinery that plays crucial roles in the disposal of misfolded proteins from the ER. Having observed poor expression for any glycoprotein ectopically coexpressed with UL148 in uninfected cells (not shown), the inventors hypothesized that UL148 might trigger the UPR. Here, the inventors show that ectopically expressed UL148 not only is sufficient to activate the PERK and IRE1 arms of the UPR but also strongly contributes to their activation during HCMV infection.

Ectopic expression of UL148 attenuates translation. As a first step to investigate whether UL148 might contribute to ER stress that would trigger the unfolded protein response (UPR), the inventors asked whether ectopic expression of UL148 in uninfected cells would dampen protein synthesis, since translational shutdown is a hallmark of stress responses, including the UPR. To address this question, the inventors employed a “Tet-on” lentiviral vector system that would allow for inducible expression of UL148 or its homolog from rhesus cytomegalovirus, Rh159, each harboring a C-terminal influenza A virus hemagglutinin (HA) epitope tag. Rh159 was used to control for any nonspecific effects of overexpression of an ER-resident glycoprotein. The inventors chose Rh159 as a control for the following reasons. First, like UL148, Rh159 is predicted to be a type I transmembrane protein with a very short cytoplasmic tail. Second, although Rh159 shares 30% amino acid identity with UL148, these two proteins reportedly carry out different functions. Third, UL148 and Rh159 are expressed at roughly similar levels during ectopic expression (see below).

Having isolated stably transduced ARPE-19 cell populations, the inventors confirmed that that anti-HA immunoreactive polypeptides of the expected size for UL148 (i148^{HA}) or Rh159 (i159^{HA}) were induced upon treatment with 100 ng/ml doxycycline (Dox). Furthermore, expression of neither protein caused any overt reduction in cell viability or number, as measured by trypan blue exclusion following 24 h of Dox induction. The inventors therefore concluded that the i148^{HA} and i159^{HA} ARPE-19 cells were suitable to address whether UL148 might affect rates of mRNA translation in metabolic labeling studies. For these experiments, i148^{HA} and i159^{HA} cells were induced (or mock induced) for transgene expression for 24 h and then incubated in the presence of ³⁵S-labeled methionine and cysteine for 30 min. In parallel, labeling was also carried out using i159^{HA} cells that were incubated in the presence of either thapsigargin (Tg) or carrier alone, so as to provide positive and negative controls, respectively, for UPR induction.

The inventors found that expression of UL148 but not Rh159 caused a substantial, ˜50% decrease in protein synthesis compared to the carrier-alone (water)-treated control, as measured by phosphorimager analysis. Strikingly, the attenuation of translation observed during UL148 expression was similar in magnitude to that seen during Tg treatment. These effects did not appear to be caused by the inducing agent, since Dox treatment of i159^{HA} cells failed to cause any reduction in ³⁵S incorporation. From these results, the inventors concluded that expression of UL148 attenuates translation. Since UL148 is an ER-resident glycoprotein with a predicted type I transmembrane topology that places most of the polypeptide in the ER lumen, it seemed plausible that the effects of UL148 on global rates of mRNA translation might be indicative of the UPR. The inventors thus sought to address the hypothesis that UL148 activates the UPR.

UL148 leads to PERK-dependent phosphorylation of eIF2α and accumulation of ATF4.Translational attenuation during the UPR is mediated by the PKR-like ER kinase PERK, which phosphorylates Ser51 of the a subunit of the ternary eukaryotic initiation factor 2 complex (eIF2α). The guanine nucleotide exchange factor eIF2B binds to the phosphorylated eIF2 complex with increased affinity and fails to exchange bound GDP for GTP. Since GDP/GTP exchange is necessary for eIF2 to participate in a new round of translational initiation and because eIF2α is present in cells at a considerable molar excess relative to eIF2B, global protein synthesis halts in response to even modest levels of phosphorylated eIF2α. Meanwhile, eIF2α phosphorylation leads to enhanced translation of certain mRNAs, such as that encoding ATF4, which harbor upstream open reading frames (uORFs) in their 5′ untranslated regions (5′UTRs) that inhibit their translation under nonstressed conditions. Although there are four different kinases that have been identified to phosphorylate eIF2α at Ser51, two observations imply that the translational attenuation the inventors observed during UL148 expression was due to activation of PERK: UL148 (i) localizes to the ER and (ii) interacts with the ERAD machinery. Therefore, the inventors next monitored levels of PERK, eIF2a phosphorylation, and ATF4 following Dox induction of either UL148 or Rh159 in ARPE-19 cells.

The inventors observed that UL148 and Rh159 proteins accumulated to readily detectable levels by 8 h after induction with Dox, although faint expression was detected at 4 h postinduction. By 48 h postinduction, the i14

8^{HA} cells showed robust levels of ATF4 protein, albeit not as high as those seen during Tg treatment, which was included as a positive control for PERK activation. Increased levels of phospho-eIF2α were detected from 24 h to 48 h following induction of UL148 but not during induction of Rh159. Moreover, PERK protein levels appeared to be upregulated at 24 h postinduction in i148^{HA} cells but not in i159^{HA} cells. Decreased mobility of the anti-PERK immunoreactive band, which likely indicates PERK autophosphorylation upon UPR activation, was readily observed in the Tg condition, but not following induction of either UL148 or Rh159, which may indicate that PERK is less synchronously activated following Dox induction of UL148 than by the comparatively shorter (4-h) Tg treatment. Although the inventors could not exclude the possibility that UL148 might cause these effects via activation of a different eIF2a kinase, the simplest interpretation of these results is that expression of UL148 activates PERK.

UL148 is sufficient to induce splicing of Xbp-1 mRNA. To determine whether UL148 activates IRE1, the inventors transfected human embryonic kidney 293T (HEK-293T) cells with plasm ids that drive expression of UL148 or Rh159 carrying C-terminal HA tags. The inventors also examined the effects of a 2-h treatment with 1 mM dithiothreitol (DTT) as a positive-control treatment known to activate IRE1. At 48 h posttransfection, the inventors harvested cells for isolation of total RNA and for protein lysates to monitor transgene expression. As a readout for IRE1 activity, the inventors used reverse transcriptase PCR (RT-PCR) to detect the removal of 26 nucleotides (nt) from the Xbp-1 mRNA. This unorthodox splicing event is catalyzed in the cytosol by IRE1; its detection is widely used as an indicator of the UPR in general and of IRE1 nuclease activity in particular. Although the inventors also tried this assay using the inventors' Dox-inducible ARPE-19 cells (not shown), the inventors found that transient transfection of HEK-293 cells gave the most readily interpretable results.

As expected, the 2-h DTT treatment caused the 26-nt intron to be spliced from nearly all the Xbp-1 mRNA detected in the inventors' assay. Cells transfected with either the Rh159 expression plasmid or empty vector failed to show notable levels of Xbp-1 splicing. In contrast, removal of the 26 bp was readily detected from cells expressing UL148, with approximately equal levels of RT-PCR products for spliced and unspliced Xbp-1. Furthermore, the expression of anti-HA-immunoreactive bands of the expected sizes for Rh159 and UL148 was confirmed by Western blotting. From these results, the inventors concluded that ectopic expression of UL148 but not Rh159 is sufficient to induce splicing of the 26-nt intron from Xbp-1 mRNA. Given that IRE1 is required for this splicing event, the inventors' results argue that UL148 is sufficient to activate IRE1.

UL148 activates IRE1 during HCMV infection. Since UL148 was apparently capable of activating the UPR when ectopically expressed, the inventors wondered whether UL148 might contribute to the UPR activation in the context of HCMV infection. Therefore, the inventors conducted a time course experiment comparing IRE1-catalyzed splicing of Xbp-1 in fibroblasts infected at a multiplicity of infection (MOI) of 1 with either wild-type (WT) HCMV strain TB40/E (TB_WT) or a UL148-null mutant, TB_148_{STOP}. Remarkably, while WT-infected cells showed increasing levels of spliced Xbp-1 (Xbp-1s) as infection progressed, UL148-null virus-infected cells showed only very low levels of spliced Xbp-1 that did not increase over time. It is also notable that during WT infection, the proportion of spliced to unspliced Xbp-1 message increased from 24 h postinfection (hpi) to 144 hpi, as these effects correlate nicely with the appearance of detectable levels of UL148 during infection and with increases in its abundance that occur as infection progresses (see below). Further, the inventors observed comparable levels of IE2 mRNA by semiquantitative RT-PCR, indicating that infection with the two viruses occurred at similar levels.

IRE1-catalyzed splicing of the Xbp-1 mRNA is expected to result in a message that can be translated into XBP1s protein. Therefore, the differences the inventors observed between WT and UL148-null infections in Xbp-1 splicing should correlate with differences in the abundance of XBP1s protein. To test this prediction, the inventors carried out Western blot experiments comparing XBP1s levels between WT and UL148-null virus-infected cells. Because IRE1 oligomerizes and undergoes transautophosphorylation in response to conditions of ER stress, the inventors also monitored IRE1 phosphorylation, making use of an antibody that is specifically immunoreactive to IRE1 polypeptides when they are phosphorylated at serine position 724 (Ser724).

WT-infected fibroblasts showed robust levels of XBP1s protein at 72 hpi and 96 hpi, comparable to those resulting from a positive-control treatment with the UPR inducer Tg. In contrast, UL148-null virus-infected cells expressed XBP1s very poorly at 72 hpi and virtually undetectably at 96 hpi. Importantly, these two time points correspond to when UL148 is expressed at peak levels during infection. Even though comparable levels of XBP1s were detected at 6 hpi and 48 hpi in both WT and UL148-null virus infection settings, the abundance of XBP1s plummeted subsequent to the 48-hpi time point during UL148-null infection, while the opposite occurred during WT infection. In tandem with the differences in XBP1s expression, detection of phospho-IRE1 at the 72-hpi and 96-hpi time points was appreciably weaker from UL148-null virus infections than from WT infections. As expected, Tg-treated fibroblast lysates showed a robust phospho-IRE1 signal that starkly contrasted to the very weak detection observed from mock-treated, noninfected cells.

The inventors interpreted these results to suggest that UL148 contributes to the activation of IRE1 during HCMV infection, which is accompanied by the concomitant cytoplasmic splicing of Xbp-1 mRNA and synthesis of XBP1s protein. Since Xbp-1 splicing is an important hallmark of UPR activation, these results also imply that UL148 is a considerable source of ER stress in the context of HCMV infected cells.

UL148 contributes to PERK-dependent increases in phosphorylated eIF2a and ATF4 during HCMV infection. To evaluate whether UL148 contributes to PERK activation during infection, the inventors monitored levels of PERK, phospho-eIF2α, and ATF4 following infection of fibroblasts with WT or UL148-null virus at an MOI of 1. The inventors found striking differences between the WT and UL148-disrupted infection contexts in each of these parameters, which together suggest a role for UL148 in activation of PERK. In WT-infected cells, ATF4 showed an obvious increase in abundance by 48 hpi and reached near-maximal expression by 72 hpi, with the highest levels being detected at 96 hpi. These changes in ATF4 expression during WT infection coincided with increased phosphorylation of eIF2a and higher levels of PERK. The kinetics of ATF4 expression were tightly correlated, to a remarkable degree, with those seen for UL148.

In cells infected with UL148-null virus, ATF4 was weakly expressed at most of the time points monitored, although faint increases were seen at 72 hpi and 84 hpi. At 96 hpi, however, a strong burst of ATF4 expression was detected, which was accompanied by an increase in phosphorylated eIF2α. This observation suggests that UL148-independent activation of one or more eIF2a kinases occurs at very late times during infection. Importantly, levels of the viral IE1 (IE1-72) protein were similar across all time points for both viruses, indicating that infection occurred efficiently in both WT and UL148-null settings, as would be expected since UL148-null mutants replicate indistinguishably from the WT in fibroblasts. Because the UL148-indepenent rise in levels of phospho-eIF2α and ATF4 occurred between 84 and 96 hpi, the inventors reasoned that the 72-hpi time point would best allow us to isolate the effect of UL148 on these indicators of PERK activation. By measuring the fluorescence signal from secondary antibodies from multiple biological replicates, the inventors were able to estimate that at 72 hpi, WT-infected cells contain 2-fold-higher levels of phospho-eIF2α (normalized to total eIF2α) and roughly 4-fold higher levels of ATF4 (normalized to beta-actin) than UL148-null virus-infected cells.

During activation of PERK under conditions of ER stress, its cytoplasmic kinase domain becomes phosphorylated. Therefore, the inventors used a commercially available phosphospecific antibody to determine whether the inventors might detect differences in phosphorylation of PERK during WT versus UL148-null infection. To maximize the sensitivity and specificity of the inventors' assay, the inventors immunoprecipitated PERK from 72-hpi cell lysates before carrying out Western blotting to detect phosphorylation of PERK at serine 713 (Ser713). The inventors consistently detected stronger immunoreactivity of an ˜150-kDa band to the phosphospecific PERK antibody in immunoprecipitate (IP) samples prepared from WT-infected cells than in those from UL148-null infections, even though the overall levels of PERK recovered in each IP reaction appeared to be roughly similar. Because the ˜150-kDa band matched the expected size of the PERK polypeptide and the relative mobility of the band detected by antibodies specific for total PERK, the inventors interpreted the result to suggest differences in phosphorylation of PERK at Ser713. This finding suggested to us that UL148 is required during HCMV infection for increases in PERK phosphorylation at Ser713.

In order to more specifically address whether PERK is required for UL148 to cause phosphorylation of eIF2a and accumulation of ATF4, the inventors used small interfering RNA (siRNA) to silence PERK expression prior to infection and then monitored for phosphorylation of eIF2a and expression of ATF4 from 24 to 72 hpi. Levels of PERK were substantially reduced but not completely eliminated by the PERK-targeted siRNA treatment, compared to the nontargeting control siRNA (NTC). During WT infection of PERK-silenced cells, phosphorylation of eIF2a was attenuated at both 48 hpi and 72 hpi, and a substantial decrease in ATF4 was seen at 72 hpi. During UL148-null infections, however, PERK knockdown led to only minimal effects on phosphorylation of eIF2a and virtually imperceptible effects on ATF4, which may well reflect reduced levels of ER stress in the absence of UL148.

Quantification of fluorescent secondary antibody signals suggested that in the case of WT virus at 72 hpi, PERK knockdown led to a 54% (2.2-fold) decrease in the level of phospho-eIF2α (normalized to total eIF2a signal) and a 33% decrease in ATF4 (normalized to beta-actin signal) and that the siRNA treatment decreased PERK expression (normalized to beta-actin) by roughly 85% during WT infection at the 72-hpi time point. Because the siRNA knockdown of PERK was incomplete, it seems likely that the inventors' results may underestimate the degree to which UL148 depends on PERK to cause phosphorylation of eIF2a and to increase ATF4 expression. Overall, the inventors interpreted these findings to argue that UL148 activates PERK during HCMV infection.

UL148 contributes to differences in mRNA levels for UPR target genes. A major function of the UPR is to cause changes in cellular gene expression. Since ATF4 and XBP1s are transcription factors that contribute to UPR-mediated changes in gene expression, the inventors next wished to determine whether UL148 contributes to the effects of HCMV on mRNA levels for cellular genes canonically regulated by the UPR. Further, because the inventors were unable to obtain an antibody sensitive enough to test whether activation of ATF6 was influenced by UL148 and because it has been reported that HCMV infection does not lead to ATF6 activation but that genes regulated by ATF6 are nonetheless upregulated, the inventors also sought to address whether UL148 might contribute to upregulation of ATF6 target genes. Therefore, the inventors isolated total RNA from WT and UL148-null virus-infected fibroblasts at 72 hpi and used reverse transcriptase-quantitative PCR (RT-qPCR) to measure mRNA levels for representative UPR target genes, including ATF6 target genes in addition to those regulated by ATF4 (PERK) and XBP1s (IRE1).

With regard to the PERK pathway, the inventors' results indicate that relative to UL148-null virus-infected cells, WT virus-infected cells on average express nearly 4-fold-higher levels of mRNA for the ATF4 target gene CHOP and roughly 2-fold-higher levels of the mRNA for another ATF4 target, GADD34. The difference in CHOP expression was found to be statistically significant (P≤0.01). Despite the UL148-dependent effects the inventors observed on IRE1 (auto)phosphorylation, Xbp-1 splicing, and XBP1s protein expression, the levels of mRNAs of XBP1s target genes did not appreciably differ between WT and UL148-null virus infections. Although the inventors cannot exclude the possibility that XBP1s target genes could be upregulated at times later than 72 hpi, the inventors' result is consistent with a previous report that failed to find an effect of HCMV-induced Xbp-1 splicing on mRNA levels for the XBP1s target gene EDEM1. Intriguingly, the inventors did find significant differences for a number of ATF6 target genes that were upregulated in WT relative to UL148-null virus infections, including BiP, PDIA4, SEL1L, and HERPUD1, all of which showed approximately 2-fold-higher expression during WT infection. Although HYOU1 was found to be 1.8-fold upregulated during WT virus infection relative to the UL148-null comparator, the difference did not reach statistical significance. From these results, the inventors concluded that UL148 contributes during HCMV infection to upregulation of UPR target genes related to the PERK and ATF6 arms of the UPR.

HCMV is estimated to carry 164 to 192 distinct genes, with a more recent study arguing for up to 751 protein-coding ORFs. Thus, the degree to which the inventors' results suggest that UL148 alone contributes to UPR induction during HCMV infection is remarkable. The original work demonstrating that HCMV activates the UPR was based on experiments using the laboratory-adapted virus strain Towne, which, unlike another widely studied laboratory strain, AD169, retains the capacity to express UL148. Accordingly, the kinetics of ATF4 protein accumulation and phosphorylation of eIF2a that the inventors observed for cells infected with wild-type (WT) strain TB40/E are highly consistent with those observed in the previous study, and the extent to which the inventors find that these indicators of PERK activation to be dampened during UL148-null virus infection is striking.

Cells infected with UL148-null viruses exhibited reduced levels of eIF2a phosphorylation and impaired induction of ATF4 at times prior to 96 hpi. Although there are three other eIF2a kinases, the inventors contend that because UL148 is an ER-resident protein and also appears to activate IRE1, another sensor of ER stress, its effects on eIF2a phosphorylation and ATF4 levels most like occur via PERK. Indeed, PERK knockdown appeared to dampen ATF4 induction at 72 hpi during WT infection, a time when its expression depends in large part on UL148. Similarly, knockdown of PERK led to attenuated levels of ATF4. Thus, both human and murine cytomegaloviruses appear to induce phosphorylation of eIF2a and ATF4 upregulation via PERK. Although MCMV does not encode a UL148 homolog, it would be interesting to know whether any single MCMV gene product contributes to PERK-mediated ATF4 upregulation in a manner comparable to that seen for UL148 in HCMV.

The inventors detected 2- to 4-fold-higher mRNA levels for two ATF4-regulated regulated genes, CHOP and GADD34, in WT compared to UL148-null virus-infected cells. Hence, the effects of UL148 on the PERK-ATF4 axis were accompanied by the expected changes in gene expression. Nonetheless, the inventors' Xbp-1 mRNA splicing results argue that UL148 is also sufficient to activate IRE1. Moreover, the stark differences the inventors observed between WT and UL148-null HCMV infections in the levels of spliced Xbp-1 (Xbp-1s) mRNA, XBP1s protein, and phosphorylated IRE1 argue that UL148 accounts for much of the IRE1 activation observed during HCMV infection during times subsequent to the onset of its expression. These effects of UL148 are particularly noteworthy as they presumably occur in the face of viral downregulation of IRE1 by the viral nuclear egress factor UL50.

Although the kinetics of Xbp-1 splicing that the inventors observed during WT infection were similar to those seen by others, the ratio of spliced to unspliced message appears to be much higher in the inventors' results, which may reflect differences in UL148 expression between strains Towne and TB40/E. In the inventors' hands, the Towne strain expresses UL148 at lower levels than TB40/E. Regardless, XBP1s target genes such as EDEM1 were not found to be upregulated in a UL148-dependent manner. Others failed to observe EDEM1 upregulation despite observing evidence of IRE1-mediated splicing of Xbp-1 during infection.

Since XBP1s target genes appear to be refractory to IRE1 activation during HCMV infection, the implications to the virus of IRE1 activation are unclear. However, splicing of Xbp-1 is not the only function of IRE1. IRE1 also activates the Jun N-terminal protein kinase (JNK) signaling pathway and degrades mRNAs associated with the rough ER. Furthermore, IRE1 confers resistance to apoptosis during hepatitis C virus infection by degrading mIR-125a. Although the inventors used Xbp-1 splicing as a specific readout for activation of IRE1, it seems conceivable that functions of IRE1 unrelated to splicing of Xbp-1 mRNA may be relevant to phenotypes governed by UL148.

Whether UL148 activates the third UPR sensor, ATF6, remains unresolved. UL148-null virus-infected cells did show lower mRNA levels for ATF6 target genes than WT-infected cells, which may suggest that ATF6 is activated by UL148. ATF6 is proteolytically processed by the same proteases that regulate sterol-responsive element binding proteins (SREPBs), S1P and S2P. Under conditions of ER stress, ATF6 transits from the ER to the Golgi apparatus, where S1P and S2P release the cytoplasmic domain of ATF6 from its transmembrane anchor, allowing it to transit to the nucleus where it binds to cis-acting ER stress regulatory elements (ERSE) and upregulates genes for ER chaperones, such as BiP (Grp78).

Nonetheless, upregulation of BiP reportedly occurs in an ERSE-independent manner during HCMV infection. Although Isler et al. were unable to detect ATF6 cleavage despite finding target genes to be upregulated during HCMV infection, the S1P/S2P processed nuclear form of ATF6, like SREBPs, is rapidly degraded in the absence of proteasome inhibitors. Hence, it is difficult to exclude the possibility that low levels of ATF6 activation occur during HCMV infection.

Why would HCMV encode a protein that activates the UPR? It is intriguing to consider why HCMV would encode a viral protein that potently triggers the UPR. Certain consequences of the UPR, such as enhanced ERAD and attenuation of translation, might be expected to be unfavorable for viral replication. For instance, degradation of mRNAs by IRE1 could hamper the expression of viral glycoproteins. Meanwhile, PERK-mediated phosphorylation of eIF2α could dampen translation of viral mRNAs while also upregulating the proapoptotic factor CHOP. Despite this, PERK is found to be required for efficient HCMV replication; in particular, defects in viral upregulation of lipid synthesis are observed during infection of cells depleted for PERK. Meanwhile, ATF6 and IRE1 are important for expansion of the ER, upregulation of ER chaperones, and increased synthesis of lipids, all of which might benefit viral replication.

Of course, maintaining translation of viral mRNAs in the face of cellular stress responses is a sine qua non for cytolytic viruses, and evasion of apoptosis is no less imperative. The literature resoundingly suggests that HCMV is no exception. Along these lines, the HCMV protein UL38 might play a particularly important role in mitigating any negative impacts on the virus of UL148-mediated activation of the UPR. UL38 disarms ER stress-mediated cell death pathways and, in a biochemically separable role maintains mRNA translation in the face of cell stress by limiting negative regulation of mTORC1 by the TSC1/2 complex. Interestingly, UL38, like UL148, both is sufficient to induce ATF4 in noninfected cells and contributes to ATF4 induction during infection. Going forward, it will be interesting to find out whether disruption of UL148 alleviates the replication defect of UL38-null viruses or, in strain AD169 (which spontaneously lost UL148 during serial in vitro passage), whether restoration of the UL148 allele exacerbates the UL38-null phenotype.

Given the substantial contribution of UL148 to UPR activation documented here and the potential for the UPR to both negatively and positively impact viral replication, it is puzzling that UL148-null viruses replicate indistinguishably from WT virus in fibroblasts. The observation that UL148 can induce substantial phosphorylation of eIF2α without causing a viral replication defect raises fascinating questions. Which viral mechanisms or gene products allow the virus to maintain efficient translation of mRNAs during UL148 expression? Does HCMV benefit from eIF2α phosphorylation? Since p-eIF2α in fact stimulates the translation of stress-regulated mRNAs such as CHOP and ATF4, what is the effect of UL148 on the host and viral proteomes during infection?

Although the inventors cannot yet exclude that decreased induction of the UPR contributes to the enhanced growth of UL148-null virus in epithelial cells, the influence of UL148 on the expression of alternative gH/gL complexes, particularly gH/gL/gO, seems a more likely explanation. A derivative of HCMV strain AD169 that was restored both for UL148 and for expression of the pentameric gH/gL/UL128-131 complex appears to replicate at least as well in epithelial cells as the parental virus lacking UL148 while failing to show differences in gH/gL/gO expression in virions. The inventors thus consider it unlikely that expression of UL148 is directly detrimental to productive replication of HCMV in epithelial cells, especially since viral factors such as UL50 and UL38 may blunt any negative impacts of UPR induction on the virus.

Implications for mechanisms underlying UL148-dependent phenotypes. Going forward, it will be beneficial to delineate which biological roles and/or phenotypic effects of UL148 require induction of the UPR and to decipher the mechanism by which UL148 triggers the UPR. The inventors cannot yet dismiss the possibility that UPR induction is incidental to the bona fide biological function(s) of UL148, which could be modulation of virion cell tropism or evasion of cell-mediated immune responses. In other words, UPR activation may not be required for the effects of UL148 that provide a fitness advantage to the virus. For example, although the HCMV ER-resident immune evasin US11 triggers the UPR in uninfected cells, the UPR does not appear to be required for US11-mediated degradation of the MHC I heavy chain. On the other hand, certain observations suggest that UPR induction may be inseparable from the role of UL148 in cell tropism. The inventors recently reported that UL148 copurifies from infected cells with SEL1L, a key component of the cellular machinery for ER-associated degradation (ERAD), and the inventors have found that UL148 attenuates ERAD of newly synthesized glycoprotein O (gO), which itself appears to be a constitutive substrate for ERAD. Therefore, one might hypothesize that UL148 interacts with the ERAD machinery to impede processing of misfolded proteins, which consequently results in UPR activation. However, the inventors cannot exclude the alternative possibility that UL148 specifically functions to activate the UPR, presumably to benefit the virus.

UL148 was recently found to block surface presentation of CD58 (LFA3), a costimulatory ligand that potentiates cytotoxic T-lymphocyte and NK-cell responses, which are likely pivotal for control of HCMV infection in vivo. Intriguingly, UL148 causes markedly reduced N-glycosylation of CD58, which is exactly the opposite of its effect on gO. Rh159, which shares significant sequence homology with UL148 and is involved in retention of a distinct set of costimulatory molecules, does not appear to activate the UPR. Although it is unknown whether UL148 requires UPR activation to downregulate CD58, knowledge of the proximal events by which UL148 activates the UPR will likely prove integral to understanding the mechanisms underlying its influence on viral immune evasion and modulation of tropism.

Finally, it is worth pointing out that UL148 may hold promise as a reagent to investigate the UPR itself. Much of the inventors' understanding of the mammalian UPR comes from experimental approaches in which toxic chemicals, such as thapsigargin or tunicamycin, are relied upon to synchronously and robustly induce the UPR in cultured cells. A recent report found that such chemicals fail to accurately recapitulate the authentic UPR induced by unfolded proteins within the ER lumen. Although the molecular events by which UL148 initiates the UPR remain to be determined, this viral ER-resident glycoprotein may represent a fascinating new tool to interrogate how cells adapt to ER stress.

Cells and virus. Primary human foreskin fibroblasts (HFF) (ATCC SCRC-1041) were immortalized by transducing lentivirus encoding human telomerase (hTERT) to yield HFFT cells, as previously described. HEK-293T cells were purchased from Genhunter Corp. (Nashville, Tenn.). The retinal pigment epithelial cell line ARPE-19 was purchased from ATCC (CRL-2302). All cells were cultured in Dulbecco's modified Eagle's medium (DMEM) (Corning 10013CV) supplemented with 25 μg/ml gentamicin, 10 μg/ml ciprofloxacin HCl, and either 5% fetal bovine serum (FBS) (Sigma-Aldrich F2442) or 5% newborn calf serum (NCS) (Sigma-Aldrich N4637).

Viruses were reconstituted by electroporation of HCMV bacterial artificial chromosomes (BACs) into HFFTs, as described previously, and grown until 100% cytopathic effect (CPE) was observed. Cell-associated virus was released by Dounce homogenization of pelleted infected cells, clarified of cell debris by centrifugation (1,000×g, 10 min), and combined with the culture supernatants containing cell-free virus. The combined cell-associated and cell-free virions were then ultracentrifuged through a 20% sorbitol cushion (85,000×g, 1 h, 4° C.). The resulting virus pellet was resuspended in DMEM containing 20% NCS.

Viruses for this study were all derived from the bacterial artificial chromosome clone of HCMV strain TB40/E, TB40E-BAC4, which was a generous gift from Christian Sinzger (University Medical Center Ulm, Ulm, Germany). A UL148-null mutant derived from TB40E-BAC4, TB_148_{STOP}, has been described elsewhere. BACs and plasmid DNAs for transfection were purified from Escherichia coli using Nucleobond Xtra Midi kits (Macherey-Nagel, Inc., catalog number 740410.50).

Virus titration. The infectivities of virus stocks and samples were determined by the 50% tissue culture infectious dose (TCID₅₀) assay. Briefly, serial dilutions of virus were used to infect multiple wells of a 96-well plate. After 9 days, wells were scored as positive or negative for CPE, and TCID₅₀ values were calculated according to the Spearman-Karber method.

Construction of plasmids. UL148 and Rh159 were PCR amplified from plasmids pEF1-UL148HA and pcDNA-Rh159 IRES-GFP (a gift from Klaus Frueh, Oregon Health Sciences University, Beaverton, Oreg.) using primer pairs UL148_reclone_Fw/UL148 reclone Rv and Rh159 Fw/Rh159_HA_Rv, respectively. The PCR product for UL148 was ligated into pcDNA3.1(+) (Invitrogen) using the BamHI and EcoRI sites, while the PCR product for Rh159 was inserted into the EcoRV site using a Gibson assembly reaction with NEB HiFi DNA assembly master mix (New England BioLabs). Final plasmids were sequence confirmed using T7 and BGH reverse primers. To construct lentiviral vectors for inducible expression of Rh159 and UL148, pinducer10-miR—RUP-PheS (a gift from Stephen J. Elledge, Harvard Medical School) (Addgene 44011) was digested with Notl and MluI to remove the miR-30 cassette and reassembled using oligonucleotide RFP_stitch in a Gibson reaction to yield pIND-RFP. Plasmid pTRE3G-dTomato was assembled by Vector Builder. The TRE3G promoter was PCR amplified with primers TRE3Gvb_Fw and TRE3Gvb_Rv and assembled into EcoRV-digested pSP72 by a Gibson reaction to yield pSP72-TRE3G, which was sequence verified using universal primer SP6. Following the example of Macias et al., the cis repressive site (crs) of the minimal CMV promoter within TRE3G was mutated from CGTTTAGTGAACCGT to CAGGTAGTGAACCGT by overlap extension PCR using primers TRE3G_crsmut_Fw and TRE3G_crsmut_Rv. Finally, the Acrs TRE3G promoter was digested out of pSP72-TRE3G using NheI/AgeI and ligated into NheI/AgeI-digested pIND-RFP to yield pOUPc-RFP. pOUPc-UL148HA was constructed by PCR amplifying the UL148HA coding DNA sequence (CDS) from plasmid pcDNA3.1-UL148HA using primers UL148HAgibs_Fw and HAgibs_Rv and Gibson assembling the product into AgeI/MluI-digested pOUPc-RFP. pOUPc-Rh159HA was constructed by PCR amplifying the Rh159HA CDS from plasmid pcDNA3.1-Rh159HA using primers Rh159HAgibs_Fw and HAeco_gibs_Rv and Gibson assembling the product into AgeI/MluI-digested pOUPc-RFP. pOUPc-UL148HA and -Rh159HA were sequence confirmed using primers CMVcrsnull_Fw and Ubc_Rv.

Lentivirus vector transduction. To generate stable i148^{HA} and i159^{HA} cell populations, replication-defective HIV-1-based lentivirus vector particles were generated from pOUPc-UL148HA or -Rh159HA, as described previously. Briefly, 5×10⁵293T cells per well of a six-well cluster plate were cotransfected with pOUPc-UL148HA or pOUPc-Rh159HA, together with psPAX2 and pMD2.G (Addgene plasmids 12260 and 12259), which were both gifts from Didier Trono (Ecole Polytechnique Federal de Laussane, Switzerland). Transfections were carried out using TranslT-293 reagent (Mirus Bio, Inc.) according to the manufacturer's instructions. Supernatants collected at 2 and 3 days posttransfection were combined, filtered through a 0.45-μm cellulose acetate syringe filter (Corning, Inc.), added to complete DMEM growth medium supplemented with 8 μg/ml Polybrene (Sigma-Aldrich), and applied to subconfluent ARPE-19 monolayers. The next day, the medium was removed and the cells were washed three times with Dulbecco's phosphate-buffered saline (PBS) (2.7 mM KCl, 1.5 mM KH₂PO₄, 137 mM NaCl, 8.1 mM Na₂HPO₄, pH 7.4). Starting at 2 days postransduction, cells were serially passaged in medium containing 2 μg/ml puromycin HCl until resistant cells grew out.

Metabolic labeling. i159^{HA} or i148^{HA} cells were seeded at 2×10⁵ cells per well in a 24-well cluster plate in Gibco Opti-MEM reduced serum medium (Thermo Fisher) supplemented with 2.5% tetracycline (Tet)-free FBS (Clontech 631101). The following day, the medium was replaced with 2.5% Tet-free FBS-Opti-MEM supplemented with either 100 ng/ml doxycycline hyclate (Dox) (Sigma-Aldrich D9891) (added from a 1,000× stock) or 0.1% (vol/vol) sterile water to control for the volume of Dox stock solution (mock induction). At 24 h postinduction, cells were washed twice in PBS supplemented with 1 mM CaCl₂) and 0.5 mM MgCl₂ and then incubated for 1 h in starving medium (DMEM lacking methionine, cysteine, and glutamine (Gibco 21013024) supplemented with 5% dialyzed FBS (Sigma F0392) and 2 mM glutamine). Cells were then pulse-labeled in starving medium containing 150 μCi/ml [³⁵S]Met/Cys (PerkinElmer NEG772) for 30 min. Dox or mock treatment was maintained throughout the starving and pulsing steps. As a positive control for translation shutdown, i159HA ARPE19 cells were treated with 2 μM thapsigargin (Sigma T9033) or 0.1% dimethyl sulfoxide (DMSO) as a carrier control at 1 h prior to Met/Cys starvation, and treatment was maintained throughout the starvation and pulse-labeling steps. Following pulse-labeling, cells were washed three times in PBS containing 1 mM CaCl₂) and 0.5 mM MgCl₂ and then immediately lysed in 2× Laemmli buffer (120 mM Tris [pH 6.8], 4% SDS, 20% glycerol, 0.02% bromophenol blue). Beta-mercaptoethanol was then added to a final concentration of 5% (vol/vol), and the samples were heated at 95° C. for 10 min. Equal volumes of lysate were resolved by sodium dodecyl sulfate-polyacrylamide gel electrophoresis (SDS-PAGE) on 12% acrylamide NuPAGE Bis-Tris precast gels (Invitrogen NP0321) according to the manufacturer's instructions. Gels were dried and exposed to a phosphor screen for 24 h before results were captured using an Amersham Typhoon IP scanner (GE Healthcare). The relative signal per lane was calculated using Bio-Rad 1-D analysis software by reading the signal volume (counts·mm²) in each lane. Lane signals were normalized to either (i) the DMSO treatment condition or (ii) each respective nontreatment condition.

Cell viability assay.i148^{HA} or i159^{HA} ARPE-19 cells (1×10⁵ per well) were seeded in a 24-well cluster plate and incubated overnight. Medium was exchanged for complete DMEM containing 100 ng/ml doxycycline and incubated for 24 h. Cells were then trypsinized, transferred to 1.5-ml microcentrifuge tubes, and spun down at 400×g for 5 min. Cell pellets were resuspended in 100 μl fresh medium and combined with 100 μl of PBS containing 0.4% trypan blue (Bio-Rad), mixed thoroughly, and counted for viability (trypan blue exclusion) and total cell number using a hemacytometer (Bright-Line).

Dox induction of UL148 and Rh159 from stably transduced ARPE-19 cells. For each well of a 24 well cluster plate, 2×10⁵ cells of iUL148 or iRh159 ARPE19 cells were seeded in 500 μl Opti-MEM medium containing 2.5% tetracycline (Tet)-free FBS (Clontech 631101). Following 24 h of incubation at 37° C., the medium was replaced with fresh 2.5% Tet-free FBS-Opti-MEM supplemented with 100 ng/ml doxycycline hyclate (Dox) (Sigma-Aldrich 9891). Where indicated, parallel wells of ARPE-19 cells were incubated for 4 h in the presence of 200 nM thapsigargin (Tg) prior to harvest. At the indicated times posttreatment, cells were washed in PBS and lysed for 1 h at 4° C. using 50 μl per well of lysis buffer (1% Triton X-100, 400 mM NaCl, 0.5% sodium deoxycholate, 50 mM HEPES, pH 7.5) supplemented with 1× protease inhibitor cocktail (Cell Signaling Technology). Lysates were collected and spun down at 18,000×g for 30 min at 4° C. Protein concentrations in supernatants were measured using the bicinchoninic acid (BCA) assay (Thermo Pierce) and normalized, and proteins were subjected to Western blotting.

siRNA treatments. HFFTs (2×10⁵ per well of a 24-well plate) were reverse transfected with 5 μmol per well of a Dharmacon siGENOME SMARTpool specific for human PERK (EIF2AK3, M004883-03-005) or with a nontargeting control SMARTpool (D-001206-14-05), using 4.5 μl of Lipofectamine RNAiMAX per well. Briefly, siRNA transfection complexes in Opti-MEM medium were added to wells prior to applying freshly trypsinized HFFTs suspended in 0.45 ml of DMEM containing 8% FBS, 25 μg/ml gentamicin, and 10 μg/ml ciprofloxacin HCl. At 24 h postseeding, cells were infected with the indicated viruses at an MOI of 1 TCID₅₀ per cell.

Xbp-1 splicing assay. HEK-293T cells were seeded into 24-well plates for overnight culture and were transfected once they reached 80 to 90% confluence using the TransiT 2020 reagent (Mirus, Inc.), with each well receiving 1 μg plasmid DNA carried by 3 μl of the transfection reagent. At 48 h posttransfection, cells were harvested and total RNA was extracted using the Qiagen RNeasy minikit as per the manufacturer's protocol, including the optional column DNase digestion step. cDNA was generated from 1 μg RNA using the qScript cDNA synthesis kit (Quantabio, catalog number 95047-100) in a 20-μl final reaction volume. One microliter of the resulting cDNA solution was then used as the template for a PCR using primers Xbp-1_FWD and Xbp-1_REV. In the context of infection, detection of IE2 (UL122) mRNA was included as indicator of HCMV infection. The IE2 primer pair was designed using PrimerQuest software (Integrated DNA Technologies, Coralville, Iowa) and includes one oligonucleotide whose priming site spans the junction of exons 3 and 5.

RT-qPCR. mRNA levels were quantified using reverse transcriptase quantitative PCR (RT-qPCR). For these experiments, 2×10⁶ HFFT cells per well were seeded in a 6-well cluster plate, incubated overnight, and subsequently infected at an MOI of 1. At 24 hpi, inocula were removed and replaced with fresh medium. At 72 hpi, total RNA was extracted using a Qiagen RNeasy minikit (Qiagen, Inc.), including the optional on-column DNase digestion step, as per the manufacturer's instructions. cDNA was generated from 1 μg RNA using the qScript cDNA synthesis kit. For each qPCR, 1 μl of cDNA was used as the template in a 15-μl final reaction volume using iQ SYBR green Supermix (Bio-Rad, Inc.) on a CFX96 real-time PCR system (Bio-Rad). mRNA levels for each gene were measured in triplicate technical replicates per biological replicate, with a total of three independent biological replicates, and the 2^{-ΔΔ}CT method was used to determine quantitative estimates of relative gene expression, with all readings being normalized to glyceraldehyde-3-phosphate dehydrogenase (GAPDH) gene transcript levels. Canonical UPR-responsive target genes were detected using previously validated qPCR primer pairs, while levels of viral IE1 (UL123) mRNA were also measured as an indicator of HCMV infection. PCR efficiencies for primer pairs ranged from 91.1% to 99.0% for the indicated UPR target genes and were 93.4% for GAPDH and 90.7% for IE1.

Statistical analyses. Statistical analyses for this study were carried out using GraphPad Prism software, version 7.0d (GraphPad, Inc., La Jolla, Calif.).

Antibodies. The following rabbit monoclonal antibodies (MAbs) from Cell Signaling Technology, (Danvers, Mass.) were used: ATF4 clone D4B8 (catalog number 11815S), PERK clone C33E10 (catalog number 3192S), PERK clone D11A8 (catalog number 5683S), phospho-eIF2α Ser51 clone D9G8 (catalog number 3398S), eIF2α clone D7D3 (catalog number 5324S), IRE1α clone 14C10 (catalog number 3294S), and XBP-1s clone D2C1F (catalog number 12782S). To detect phosphorylation of PERK, rabbit phospho-PERK (Ser713) antibody (catalog number 649402; BioLegend, San Diego, Calif.) was used. To detect phosphorylation of IRE1a, phospho-IRE1α (Ser724) polyclonal antibody (catalog number PA1-16927; Invitrogen) was used. HCMV IE1 was detected using mouse MAb clone 1B12 (a gift from Thomas Shenk, Princeton University), and beta-actin was detected using a rabbit MAb (catalog number 926-42210; Li-Cor Biosciences, Inc.). A rabbit polyclonal anti-HA epitope antibody (Bethyl Laboratories, Inc., Montgomery, Tex.) and a previously described rabbit polyclonal serum specific for UL148 were also used.

Western blotting and IP. For detection of all proteins other than phospho-Ser51 eIF2α (see below), Western blotting was carried out as previously described. Briefly, cells were lysed at 4° C. for 1 h in lysis buffer (1% Triton X-100, 400 mM NaCl, 0.5% sodium deoxycholate, and 50 mM HEPES [pH 7.5] supplemented with 1× protease inhibitor cocktail [Cell Signaling Technology]). For experiments in which phospho-specific antibodies were used [phospho-IRE1a(Ser724), phospho-PERK(Ser713), or phospho-eIF2α(Ser51)], lysis buffer was further supplemented with 1× phosphatase inhibitor cocktail (catalog number 5870S; Cell Signaling Technology). Cell lysates were clarified by centrifugation at 18,000×g for 30 min at 4° C., combined with an equal volume of 2× Laemmli buffer containing 10% beta-mercaptoethanol, and heated at 85° C. for 10 min prior to being resolved by SDS-PAGE on 10% acrylamide gels and transferred to nitrocellulose membranes (Whatman Protran, 0.45-μm pore size). Efficient transfer was confirmed using Ponceau S staining (not shown). All subsequent blocking, washes, and incubation steps were performed with gentle rocking. Membranes were blocked using a solution of 5% powdered milk (PM) in PBS containing 0.01% Tween 20 (PBST) (PM-PBST). Unless otherwise noted, all antibodies were applied to membranes in PM-PBST as a 1:1,000 dilution (or, for IE1 MAb, a 1:200 dilution) of the hybridoma supernatant and incubated overnight at 4° C. or for 1 h at room temperature. Following three 5-min washes in 1×PBS, IRDye-800-conjugated donkey anti-rabbit or anti-mouse secondary antibodies (Li-Cor, Inc.) were applied at 1:10,000 in PM-PBST and incubated for 1 h. After 3 washes in PBST, immunoreactive polypeptides were detected and, where applicable, quantified using a Li-Cor Odyssey imager (Li-Cor Biosciences). For detection of Ser51-phosphorylated eIF2α, a protocol from the laboratory of David Ron (Cambridge Institute for Medical Research, United Kingdom) was used. The differences from the inventors' standard procedures were as follows. Membranes were blocked for 2 h at room temperature in a solution of 5% bovine serum albumin (BSA) in PBS containing 0.01% Tween 20, followed by a second 10-min blocking step in PM-PBST. Following three washes in PBS, membranes were incubated overnight in a solution of phospho-eIF2α antibody diluted 1:1,000 in PBS supplemented with 5% BSA.

For immunoprecipitation (IP) of PERK, approximately one million HFFTs were infected with either TB_WT or TB_148_{STOP} virus. At 72 hpi, each set of infected HFFT cells were washed once with PBS at room temperature and then lysed in 200 μl of ice-cold lysis buffer (see above) supplemented with 1× phosphatase inhibitor cocktail (Cell Signaling Technology). Lysates were clarified by centrifugation at 18,000×g for 30 min at 4° C. Four microliters of anti-PERK clone D11A8 antibody (Cell Signaling Technology) was then added to 200 μl of each lysate (TB_WT or TB_148_{STOP}) and rotated in a microcentrifuge tube at 4° C. for 4 h. Subsequently, 25 μl of protein G magnetic bead slurry (EMD Millipore, catalog number LSKMAGG10) was added to each IP reaction mixture, and the mixtures were then allowed to rotate overnight at 4° C. Beads were then washed three times in lysis buffer prior to elution of proteins via heating at 50° C. in 2× Laemmli buffer containing 10% beta-mercaptoethanol.

KITS: Any of the embodiments of the invention described herein can be used together with a set of instructions, i.e., to form a kit. The kit may include instructions for use of the ISR activity scanner as described herein, along with cells that induceably express 148-marker, along with the inducement chemical. For example, the kit may include ARPE-19 epithelial cells carrying an doxycycline inducible gene expression cassette encoding UL148-GFP and doxycycline hyclate.

The invention illustratively disclosed herein suitably may explicitly be practiced in the absence of any element which is not specifically disclosed herein. While various embodiments of the present invention have been described in detail, it is apparent that various modifications and alterations of those embodiments will occur to and be readily apparent those skilled in the art. However, it is to be expressly understood that such modifications and alterations are within the scope and spirit of the present invention, as set forth in the appended claims. Further, the invention(s) described herein is capable of other embodiments and of being practiced or of being carried out in various other related ways. In addition, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items while only the terms “consisting of” and “consisting only of” are to be construed in the limitative sense.

Wherefore, I/we claim:

1. A method of detecting integrated stress response activity in living cells comprising:
inducing UL148-marker expression;
scanning the cells to determine if the UL148 is punctate or diffuse; and
ranking the level of ISR activity based on how punctate the UL148 is in the cell.

2. The method of claim 1 wherein the marker is a fluorescent marker.

3. The method of claim 2 wherein the fluorescent marker is Green fluorescent protein.

4. The method of claim 1 wherein the cell is an epithelial cell.

5. The method of claim 1 wherein the cells are ARPE-19 epithelial cells carrying a doxycycline inducible gene expression cassette encoding UL148-GFP and treating the cells for 18 hours with 100 ng/mL doxycycline hyclate to induce UL148-GFP expression.

6. The method of claim 1 wherein the cells have an inducible gene expression cassette encoding UL148-marker expression.

7. The method of claim 1 wherein the UL148-marker expression is induced by the cells being treated with an antibiotic.

8. The method of claim 1 further comprising adding marker to the cells.

9. The method of claim 1 wherein the cells are mutated to express UL148-marker upon inducement.

10. A kit to conduct the method of claim 1.
